(19) United States
(12) Patent Application Publication
(10) Pub. No.: US 2024/0377416 A1
(43) Pub. Date: Nov. 14, 2024

(54) Method for Using a Marker Set for Determining the Risk of Kidney Rejection

(71) Applicant: Numares AG, Regensburg (DE)

(72) Inventors: Johannes Eiglsperger, Walkenstetten (DE); Sindy Neumann, Regensburg (DE); Philipp Pagel, Tegernheim (DE); Maximilian Zucker, Muenchen (DE); Volker Pfahlert, Kandern (DE)

(21) Appl. No.: 18/776,680

(22) Filed: Jul. 18, 2024

Related U.S. Application Data
(63) Continuation of application No. 16/490,739, filed on Sep. 3, 2019, now abandoned.

(30) Foreign Application Priority Data
Mar. 15, 2017 (DE) 102017105566.5

(51) Int. Cl. G01N 33/68; G01N 33/70

(52) U.S. Cl. CPC G01N 33/6893; G01N 33/70; G01N 2800/245; G01N 2800/50

(57) ABSTRACT

Provided is an in vitro method for determining the risk of kidney rejection of an individual after having obtained a kidney donation using a marker set including at least three substances chosen from the group consisting of 3-hydroxy isovalerate, acetyl carnitine, alanine, citrate, dimethylamine, glucose, glucuronate, hippurate, lactate, malonate, methyl guanidine, methyl malonate, methyl succinate, p-cresol, phenyl acetate, phenylacetylglycine, phenylacetylglutamine, taurine, trigonelline, and urea. The marker set also includes at least one substance chosen from the group consisting of alanine, dimethylamine, glucuronate, phenylacetylglutamine, and urea.

### CROSS-REFERENCE TO RELATED APPLICATIONS

This application is a continuation of U.S. patent application Ser. No. 16/490,739, filed on Mar. 14, 2018, which is the United States national phase of International Application No. PCT/EP2018/056414 filed Mar. 14, 2018, and claims priority to German Patent Application No. 10 2017 105 566.5 filed Mar. 15, 2017, the disclosures of which are hereby incorporated by reference in their entirety.

### BACKGROUND OF THE INVENTION

### Field of the Invention

The disclosure relates to an in vitro method for determining the risk of kidney rejection of an individual after having obtained a kidney donation using a marker set, to a marker set and to a method for analyzing an isolated body fluid sample in vitro.

### Description of Related Art

It is of high medical interest to determine the risk of kidney rejection after kidney transplantation by an easily applicable method. For this purpose, WO 2012/045773 A9 discloses certain substances that are well suited for determining the according risk of kidney rejection.

Also U.S. Pat. No. 9,541,620 B2 discloses certain substances that might be used for determining the risk of kidney rejection. Thereby, it focuses on trimethylamine-N-oxide (TMAO) as most potent biomarker.

WO 2002/010755 A2 describes that increased levels of creatinine or urea in the blood serum are an indication for a failure of a kidney transplant.

Malyszko et al. describe in a publication that serum creatinine level-though connected to significant disadvantages-still remains the “gold standard” in assessment of kidney function (Malyszko, Jolanta, et al. “Biomarkers of delayed graft function as a form of acute kidney injury in kidney transplantation.” Scientific Reports 5 (2015): 11684).

### SUMMARY OF THE INVENTION

It is an object underlying the proposed solution to provide additional marker substances and appropriate marker combinations suited to determine the risk of kidney rejection and to enhance the overall performance of an according analysis method.

This object is achieved by the method for using a novel marker set as described herein, i.e., an in vitro method for determining (predicting) the risk of kidney rejection of an individual after having received a kidney donation. While according to prior art techniques generally individual substances are used as marker, the inventors found out that a sufficiently high accuracy of an according analysis method can only be achieved if at least three substances are used as markers. Thus, the instantly claimed use of a marker set requires the presence of at least three substances to be included in the marker set. These substances are to be chosen from the group consisting of 3-hydroxy isovalerate, acetyl carnitine, alanine, citrate, dimethylamine, glucose, glucuronate, hippurate, lactate, malonate, methyl guanidine, methyl malonate, methyl succinate, p-cresol, phenyl acetate, phenylacetylglycine, phenylacetylglutamine, taurine, trigonelline, and urea.

Some of these substances are already disclosed as suited markers for determining the risk of kidney rejection after kidney transplantation in WO 2012/045773 A9, namely 3-hydroxy isovalerate, acetyl carnitine, citrate, glucose, hippurate, lactate, malonate, methyl guanidine, methyl malonate, methyl succinate, p-cresol, phenyl acetate, phenylacetylglycine, taurine, and trigonelline. The inventors surprisingly found out that the accuracy of an according analysis methods can be improved if at least one of certain additional substances is necessarily present in the marker set. Notably, these substances are alanine, dimethylamine (DMA), glucuronate, phenylacetylglutamine (PAQ), and urea. Therefore, the marker set the use of which is instantly claimed, necessarily comprises at least one substance of the group consisting of alanine, dimethylamine, glucuronate, phenylacetylglutamine, and urea. These substances will also referred to in the following as newly identified marker substances. The higher performance and thus better suitability of the marker set comprising at least one of these newly identified marker substances will be explained in more detail with respect to the exemplary embodiments.

It is possible that only one of the newly identified marker substances is present in the marker set. It is likewise possible that two, three, four, five, or six newly identified marker substances are present in the marker set. It is also possible that the marker set exclusively comprises newly identified marker substances.

The individual substances being present in the marker set are used such that the concentration of these substances in a sample is determined. Then, the substance concentration can be used to calculate a score. The score is indicative for the risk of kidney rejection of an individual who previously underwent a kidney transplantation, i.e. who previously received a kidney donation.

In an embodiment, it is possible to define certain groups of risk for rejecting a kidney after kidney transplantation. Thus, it is possible to define a group of high-risk, a group of intermediate risk, a group of low risk and a group of very low risk in order to classify the determined or predicted risk of kidney rejection in an easily accessible way. It is also possible to use only some of these groups (e.g., high risk, intermediate risk, low risk; or high risk, low risk, very low risk) or to define additional groups.

When defining these groups (in particular on the basis of training data set) for a specific model making use of certain marker substances, it is suited to define the high-risk group such that at least 40%, in particular at least 45%, in particular at least 50% of the samples assigned to this group are samples from individuals that showed a kidney rejection. In an embodiment, the group of intermediate risk is defined such that 8 to 20%, in particular 10 to 17%, in particular 11 to 15% of all samples assigned to this group are samples from individuals that encountered a kidney rejection. In an embodiment, the group of low risk is defined such that 2 to 5%, in particular 2.5 to 4.5, in particular 3 to 4% of all samples assigned to this group are samples from individuals that encountered a kidney rejection. In an embodiment, the group of very low risk is defined such that not more than 1.5%, in particular not more than 1%, in particular not more than 0.5% of all samples assigned to this group are samples from individuals that encountered a kidney rejection. By such a grouping, a calculated score can be easily assigned to one of these groups so that the medical relevance of the calculated score is easily accessible.

In an embodiment, the marker set comprises 1, 2, 3, 4, 5, 6, 7, 8, 9, 10, 11, 12, 13, 14, 15, 16, 17, 18, 19, 20 or 21 individual substances. As already explained, 1, 2, 3, 4, 5 or 6 substances of the group consisting of alanine, dimethylamine, glucuronate, phenylacetylglutamine, and urea can be present in the marker set, wherein at least one of these substances is necessarily present in the marker set.

In an embodiment, the marker set necessarily comprises at least one substance chosen from the subgroup of newly identified marker substances consisting of alanine, dimethylamine, glucuronate, and urea. These newly identified marker substances have been proven to be especially suited for enhancing the overall performance of the marker set for determining the risk of kidney rejection.

In another embodiment, the marker set necessarily comprises urea. Urea turned out to be one of the most suited substances to increase the overall performance of the marker set.

In a further embodiment, the marker set comprises necessarily urea and alanine. In another embodiment, the marker set comprises necessarily urea and glucuronate. In a further embodiment, the marker set necessarily comprises urea, alanine and glucuronate. In a further embodiment, the marker set comprises necessarily urea, alanine, and dimethylamine.

In an embodiment, the marker set comprises lactate and citrate as already principally known suited marker substances in coming nation with one marker substances chosen from the group consisting of alanine, dimethylamine, glucuronate, phenylacetylglutamine, and urea.

In an embodiment, the marker set does not comprise dimethylamine, lactate, acetate and alanine at the same time.

In an embodiment, the marker set does not comprise only dimethylamine, lactate and acetate.

In an embodiment, the marker set does not comprise only dimethylamine, lactate and alanine.

In an embodiment, the marker set does not comprise only dimethylamine, acetate and alanine.

In an embodiment, the marker set does not comprise only lactate, acetate and alanine.

The following examples of marker sets are particularly suited marker sets:
Lactate, citrate and urea.
Lactate, citrate, urea and alanine.
Lactate, citrate, urea and glucuronate.
Lactate, citrate, urea, alanine and glucuronate.
Lactate, citrate, urea, alanine and dimethylamine.

The solution relates in an aspect also to a method for using the same marker set for diagnostics of the risk of kidney rejection of an individual after having received a transplanted kidney. Thus, in this aspect, the solution relates to a marker set comprising at least three substances chosen from the group consisting of 3-hydroxy isovalerate, acetyl carnitine, alanine, citrate, dimethylamine, glucose, glucuronate, hippurate, lactate, malonate, methyl guanidine, methyl malonate, methyl succinate, phenyl p-cresol, acetate, phenylacetylglycine, phenylacetylglutamine, taurine, trigonelline, and urea, for use in diagnostics of the risk of kidney rejection of an individual after having received a kidney donation, with the provision that the marker set comprises at least one substance chosen from the group consisting of alanine, dimethylamine, glucuronate, phenylacetylglutamine, and urea.

Embodiments of the in vitro method for using the marker set explained above are likewise applicable to an in vivo use or the diagnostic use of the marker set in any desired combination.

The claimed method for using the marker set in diagnostics constitutes another medical indication of this marker set. Thereby, at least one of the newly identified marker substances, namely one of alanine, dimethylamine, glucuronate, phenylacetylglutamine, and urea, is always present in the marker set.

In an aspect, the solution also relates to method of analyzing an isolated body fluid sample in vitro. This method comprises the steps explained in the following. In a first step, a body fluid sample from an individual who has received a kidney donation is provided. This body fluid sample can be taken at any time after kidney transplantation, e.g., 0 days to 400 days after transplantation, in particular 1 day to 365 days after transplantation, in particular 2 days to 350 days after transplantation, in particular 3 days to 340 days after transplantation, in particular 4 days to 330 days after transplantation, in particular 5 days to 320 days after transplantation, in particular 6 days to 310 days after transplantation, in particular 7 days to 300 days after transplantation, in particular 8 days to 290 days after transplantation, in particular 10 days to 280 days after transplantation, in particular 11 days to 270 days after transplantation, in particular 12 days to 260 days after transplantation, in particular 13 days to 250 days after transplantation, in particular 14 days to 240 days after transplantation, in particular 15 days to 230 days after transplantation, in particular 16 days to 220 days after transplantation, in particular 17 days to 210 days after transplantation, in particular 18 days to 200 days after transplantation, in particular 19 days to 190 days after transplantation, in particular 20 days to 180 days after transplantation, in particular 25 days to 170 days after transplantation, in particular 30 days to 160 days after transplantation, in particular 40 days to 150 days after transplantation, in particular 50 days to 140 days after transplantation, in particular 60 days to 130 days after transplantation, in particular 70 days to 120 days after transplantation, in particular 80 days to 110 days after transplantation, in particular 90 days to 100 days after transplantation. Thereby, “0 days” means less than 24 hours after kidney transplantation.

Afterwards, the concentration of at least three substances chosen from the group consisting of 3-hydroxy isovalerate, acetyl carnitine, alanine, citrate, dimethylamine, glucose, glucuronate, hippurate, lactate, malonate, methyl guanidine, methyl malonate, methyl succinate, p-cresol, phenyl acetate, phenylacetylglycine, phenylacetylglutamine, taurine, trigonelline, and urea, is determined with a suited measuring technique. These substances serve as marker substances. Thereby, the concentration of at least one substance chosen from the group consisting of alanine, dimethylamine, glucuronate, phenylacetylglutamine, and urea is necessarily determined.

A very well suited measuring technique for determining the concentration of the individual substances is nuclear magnetic resonance spectroscopy (NMR spectroscopy).

The determined concentrations are afterwards used to calculate a score. Thereby, the score is indicative for the risk of kidney rejection of the individual.

It is generally possible to calculate the score in any desired way. A suited way to calculate the score is disclosed on pages 25 to 27 of WO 2012/045773 A9. Another suited way to calculate the score is the following:

$$score = \frac{1}{1 + {e}^{- ω}} ,$$

wherein

$$ω = a + {\sum }_{x = 1}^{n} {b}_{x} \cdot {I}_{x}$$

a=const.
bₓ=substance specific coefficient
I=parameter being indicative for the concentration of substance x

Thereby, the individual factors a, b need to be adjusted according to the underlying model and can vary in dependence on the specific substances considered in the marker set. Parameter “I” can be, e.g., the signal intensity of an according signal observed in the evaluated measuring result. To give an example, “I” can be the signal intensity of an NMR signal in an NMR spectrum if NMR spectroscopy is used as measuring technique.

In an embodiment, the calculated score is output and presented to the individual and/or to a third person such as a physician or medical staff. The output can be performed on a display (i.e., in an electronic way) or in printed form. Thereby, it is also possible to generate a report indicating the score, optionally in combination with a comparative scale of possible scores and their meaning with respect to the risk of kidney rejection after kidney transplantation.

In an embodiment, the sample is grouped into one of at least two predefined groups on the basis of the calculated score. The according grouping can also be indicated on an according report.

In an embodiment, the body fluid sample is a urine sample or a blood sample. Thereby, the term “blood” relates to whole blood, to blood serum, to blood plasma or to any other blood preparation derivable from whole blood or from other blood preparations.

Using a urine sample as body fluid sample is particularly suited for carrying out the method since urine samples are easily collectable from individuals and do not need any closer interaction with the body of the individual.

To compensate for differing overall marker concentrations in different samples, the determined concentrations are, in an embodiment, normalized to the additionally determined concentration of creatinine. This is especially suited in case of urine samples. If a person, from whom the analyzed sample has been obtained, has ingested a big volume of liquid, his or her total urine volume is also big. At the same time, the overall concentration of the marker substances (and other substances) in the urine is in such a case generally lower than in a sample taken from a person who has ingested a lower volume of liquid and has a lower total urine volume. If the determined marker concentrations are divided by the creatinine concentration in the same urine sample (i.e., normalized to the creatinine concentration), these effects are erased.

The score referred to above can form the basis of a diagnostic test being based on at least three metabolites.

In an embodiment, an extended score is calculated starting from the score by additionally considering at least one further value. This can further increase the performance of a diagnostic test being based on the instantly described method. This further value is chosen from the group consisting of a value being indicative for the concentration of creatinine in blood serum of the individual, a value being indicative for the glomerular filtration rate (GFR) of the individual, and a value being indicative for the estimated glomerular filtration rate (eGFR) of the individual. Thereby, it is particularly suited to calculate an extended score by considering a value being indicative for the concentration of creatinine in blood serum if a urine sample of the individual is analyzed. In doing so, both information relating to the concentration of individual marker substances in the urine of the individual (patient) as well as information relating to the concentration of creatinine in the blood serum of the individual are taken into account. This additionally enhances the reliability of the whole method with respect to the correctness of the calculated extended score.

The combination of the score based on metabolites and additional information based on creatinine concentration in blood serum, GFR and/or eGFR results in a decision support system. Such a decision support system is particularly suited to group different samples into different groups (such as different risk groups for a specific disease or indication like the risk of kidney rejection after kidney transplantation of an individual who has received a kidney donation). Based on such a grouping, a physician can be assisted in assessing the medical relevance of the obtained result or in making a diagnosis.

The described in vitro method can also be regarded as a method for providing original data that can afterwards be used by a physician to make a diagnosis regarding the risk of kidney rejection after kidney transplantation. Generally, this diagnostic step does not form part of the claimed method. However, in an embodiment, the method can encompass such a diagnostic step.

In a further aspect, the solution relates to a method for assessing or predicting the risk of kidney rejection after kidney transplantation of an individual who has obtained a kidney donation. This method comprises the following steps:

In a first step, the concentration of at least three substances chosen from the group consisting of 3-hydroxy isovalerate, acetyl carnitine, alanine, citrate, dimethylamine, glucose, glucuronate, hippurate, lactate, malonate, methyl guanidine, methyl malonate, methyl succinate, p-cresol, phenyl acetate, phenylacetylglycine, phenylacetylglutamine, taurine, trigonelline, and urea is determined in a body fluid sample of an individual who has obtained a kidney donation. This is done by analyzing the body fluid sample in the presence or absence of the individual with a suited measuring technique, with the provision that the concentration of at least one substance chosen from the group consisting of alanine, dimethylamine, glucuronate, phenylacetylglutamine, and urea is determined.

In a second step, a score is calculated from the determined concentrations, wherein the score is indicative for the risk of kidney rejection of the individual.

Afterwards, the specific risk of kidney rejection is derived from the calculated score based on a predefined allocation of individual scores to specific risks of kidney rejection. Thereby, the calculated score can be grouped into one of different predefined groups being indicative for different risks of kidney rejection.

This risk of kidney rejection is then output to the individual or third person like a physician or medical staff.

In an embodiment, the risk of kidney rejection is determined on an extended score as explained above. The method then forms part of a decision support system or makes up such a decision support system.

In an aspect, the solution relates to a decision support system implementing a method as explained above.

In an aspect, the solution relates to a method for using the creatinine concentration in a urine sample for normalizing the concentration of at least one other substance determined in the same urine sample, in particular by dividing the concentration of the other substance by the concentration of creatinine. This normalization method forms an individually claimed part of the instant disclosure that can, in an embodiment, combined with any other method or use described or claimed herein.

Thereby, all embodiments explained with respect to the method for analyzing an isolated body fluid sample can be likewise applied in any desired combination to the described method for assessing the risk of kidney rejection. This is especially true for the output report explained above in connection to the in vitro method for analyzing an isolated body fluid sample.

All explained embodiments of the described uses can be applied in any desired combination to the described methods, and vice versa.

### BRIEF DESCRIPTION OF THE DRAWINGS

Aspects of the solution will now explained in more detail with respect to embodiments and accompanying Figures.

FIG. 1A shows a receiver operating characteristic (ROC) plot of sensitivity versus specificity of a model for determining the risk of kidney rejection taking into account lactate, citrate and trigonelline as marker substances (comparative example);

FIG. 1B shows an ROC plot of sensitivity versus specificity of a model for determining the risk of kidney rejection taking into account lactate, citrate and hippurate as marker substances (comparative example);

FIG. 1C shows an ROC plot of sensitivity versus specificity of a model for determining the risk of kidney rejection taking into account lactate, citrate and urea as marker substances;

FIG. 2A shows an ROC plot of sensitivity versus specificity of a model for determining the risk of kidney rejection taking into account lactate, citrate, trigonelline and glucose as marker substances (comparative example);

FIG. 2B shows an ROC plot of sensitivity versus specificity of a model for determining the risk of kidney rejection taking into account lactate, citrate, hippurate and glucose as marker substances (comparative example);

FIG. 2C shows an ROC plot of sensitivity versus specificity of a model for determining the risk of kidney rejection taking into account lactate, citrate, trigonelline and hippurate as marker substances (comparative example);

FIG. 2D shows an ROC plot of sensitivity versus specificity of a model for determining the risk of kidney rejection taking into account lactate, citrate, urea and alanine as marker substances;

FIG. 2E shows an ROC plot of sensitivity versus specificity of a model for determining the risk of kidney rejection taking into account lactate, citrate, urea and glucuronate as marker substances;

FIG. 2F shows an ROC plot of sensitivity versus specificity of a model for determining the risk of kidney rejection taking into account lactate, citrate, urea and alanine as marker substances and additionally considering the concentration of creatinine in blood serum of the individual;

FIG. 2G shows an ROC plot of sensitivity versus specificity of a model for determining the risk of kidney rejection taking into account lactate, citrate, urea and alanine as marker substances and additionally considering the estimated glomerular filtration rate of the individual;

FIG. 3A shows an ROC plot of sensitivity versus specificity of a model for determining the risk of kidney rejection taking into account lactate, citrate, trigonelline, hippurate and glucose as marker substances (comparative example);

FIG. 3B shows an ROC plot of sensitivity versus specificity of a model for determining the risk of kidney rejection taking into account lactate, citrate, urea, alanine and glucuronate as marker substances; and

FIG. 3C shows an ROC plot of sensitivity versus specificity of a model for determining the risk of kidney rejection taking into account lactate, citrate, urea, alanine and dimethylamine as marker substances.

### DESCRIPTION OF THE INVENTION

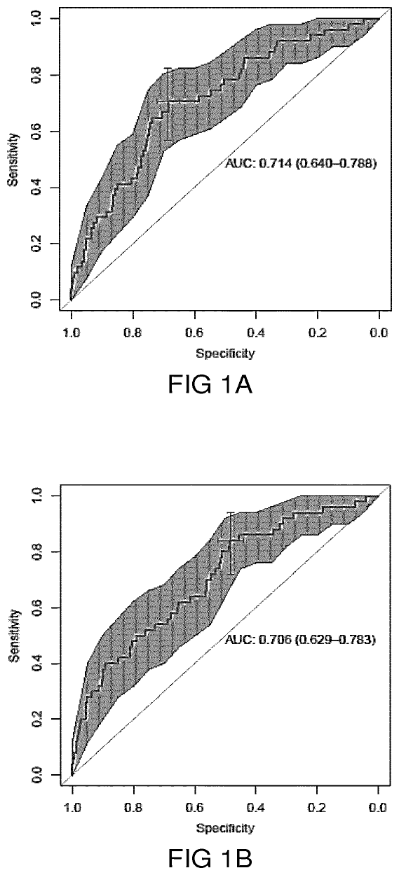

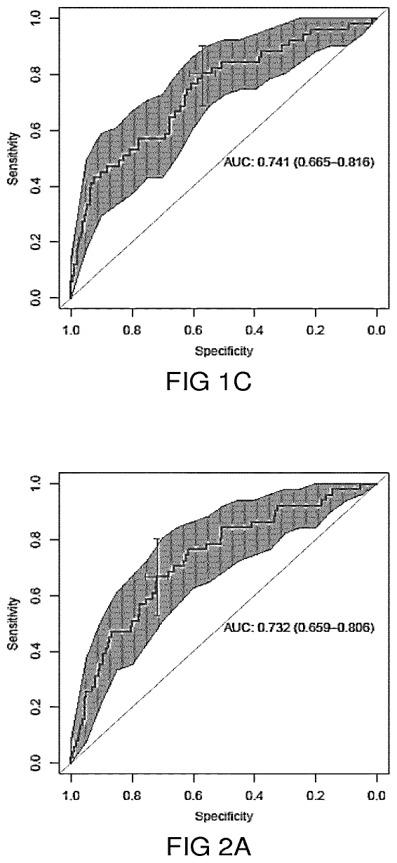

The results depicted in FIGS. 1A to 3C are based on an analysis of more than 4200 urine samples from patients having obtained a kidney donation. The urine samples were taken from these patients in a time window between 14 days after transplantation and 365 days after transplantation. Thereby, in a first step a training sample set (based on more than 870 samples) has been analyzed to identify suited marker substances. Subsequently, the identified suited marker substances have been tested on a validation sample set (more than 580 samples) to assess the performance of a determination of the risk of kidney rejection making use of these marker substances.

### Sample Handling and Preparation

Mid-stream urine samples were collected in standard plastic urine cups and aliquots of 1 ml were transferred into 1.5 ml sample tubes. The aliquots were frozen at −20° C. within 8 hours after collection. For storage, the tubes were kept at −80° C. For NMR measurements, aliquots were allowed to thaw at room temperature. Upon complete thawing, 600 μl of the aliquot were mixed with 120 μl of Axinon® urine additive solution. The mixture was centrifuged at 20000 g for 10 min and 600 μl of the supernatant was transferred to 5 mm NMR tubes and kept at 4-8° C. until measurement.

### NMR Measurements

All measurements were carried out on a Bruker Avance II+600 MHz NMR spectrometer using a PATXI 1H/D-13C/15N Z-GRD probe. All samples were kept at 5-7° C. in the SampleJet and brought to the target temperature in the integrated preheating block before measurement. A standard pulse program with 30 degree excitation pulse and pre-saturation for water suppression was used (zgpr30).

Samples were measured in batches of up to 93 samples per run. In addition to the analytical samples, each run included one Axinon® urine calibrator sample and two Axinon® urine control samples (before and after the analytical urine samples, respectively) in order to assure ideal measurement conditions throughout the run.

### Signal Analysis

NMR spectra underwent automatic referencing, phase correction and baseline correction before further analysis.

Subsequently, the NMR spectra underwent an automatic standardization and calibration procedure to minimize between-device, between-day and between-run effects. The quality of each of these spectra was assessed by a custom spectrum qualification algorithm that analyzes general spectral properties, e.g., offset and tilt of the baseline in selected spectral regions, and properties of selected indicator signals, e.g., signal position, shape and width. Spectra that did not meet the predefined quality criteria were excluded from further analysis.

Successfully qualified spectra were divided into 403 bins of 0.04 ppm width positioned with 50% overlap between consecutive bins covering a spectral range from 0.96 to 9.04 ppm. The area from 4.5 to 5.0 ppm (water signal) was not considered in the analyses. Thus, a total of 377 bins for subsequent analysis remained.

Quantification of specific signal peaks was done by fitting Pseudo-Voigt functions, which represent a linear combination of a Gaussian and a Lorentzian function, to each peak of interest. The resulting signal fits were checked for goodness of fit in order to reject results of insufficient fit quality.

### Marker Substance Identification

Suited marker substances were identified with the aid of the obtained NMR spectra in an iterative way by combining the NMR information with the additional information whether the underlying sample was taken from an individual that showed kidney rejection or from an individual that did not show kidney rejection.

An individual who recovered well from kidney transplantation and did not show any significant abnormality was considered to be an individual that did not show kidney rejection. Individuals encountering kidney problems after kidney transplantation were subjected to a biopsy being the gold standard for determining whether or not the donated kidney is rejected. If the outcome of such a biopsy was that kidney rejection has been taken place or was about to take place, these individuals were considered to be individuals showing kidney rejection.

Since a biopsy can cause hematuria and may potentially impact kidney function directly, only urine samples that have been taken within max. 7 days prior to biopsy were included in the assessment. Thus, if a urine sample was taken from an individual that subsequently underwent a biopsy which resulted in the finding that a kidney rejection was about to take place or has been taken place, the sample was assigned to an individual having a high risk of kidney rejection. If a biopsy did not provide any result indicative for kidney rejection, the according individual was considered to be a person having no significant risk of kidney rejection.

### Test of Identified Marker Substances

The identified marker substances were tested in different combinations to assess their suitability for determining the risk of kidney rejection of an individual after kidney transplantation. In doing so, the result of the determination based on the marker substances (predicted risk) has been checked against clinical signs of kidney rejection and/or transplant dysfunction as well as the results of performed biopsies (histologically proven kidney rejection).

The results are summarized in receiver operating characteristic (ROC) plots. In these plots, the area under the curve (AUC) indicates the fitness of the prediction. If the AUC is 0.5, the prediction is to be considered random and thus not well suited. The higher the AUC, the better is the prediction model.

FIG. 1A shows an ROC plot of a model for determining the risk of kidney rejection taking into account lactate, citrate and trigonelline as marker substances (comparative example). These substances are already known from prior art to be used as substances for determining the risk of kidney rejection, but have not been described so far in this specific combination. To compensate for concentration differences, all substance concentrations determined in this and in the following examples have been normalized to the concentration of creatinine in urine. Thus, the concentration of creatinine in urine serves as internal standard. The resulting AUC is 0.714. This AUC is much better than a simply random prediction and also better than an according AUC of an individual substance used as marker for determining the risk of kidney rejection. Nonetheless, a further enhancement of the reliability of the prediction model is desired.

If trigonelline is replaced by hippurate, the AUC is 0.706 and thus even lower (comparative example) as can be seen from FIG. 1B. Hippurate is also already known from prior art to be used as marker for determining the risk of kidney rejection.

If, however, trigonelline is replaced by a newly identified marker substance, namely, urea, the AUC significantly increases to 0.741. This is shown in FIG. 1C. Thus, a combination of two “old” marker substances (lactate and citrate) with a newly identified marker substance (urea) results in a much higher performance of the respective prediction model.

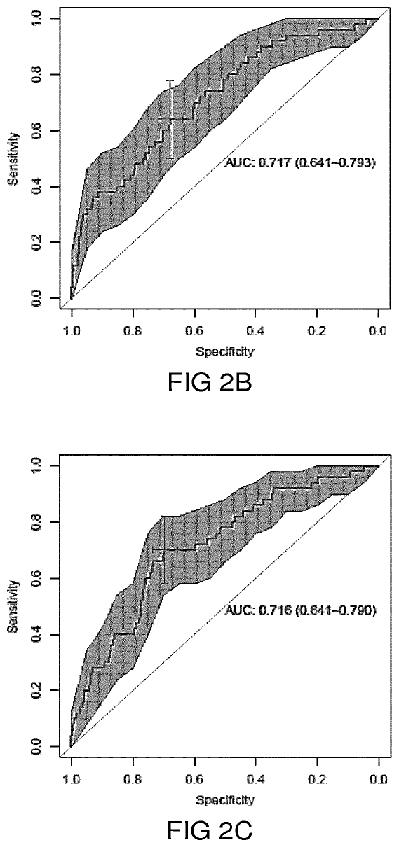

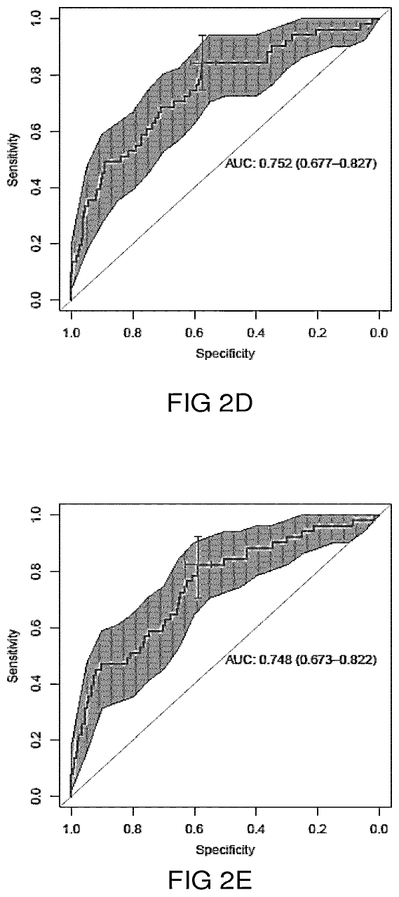

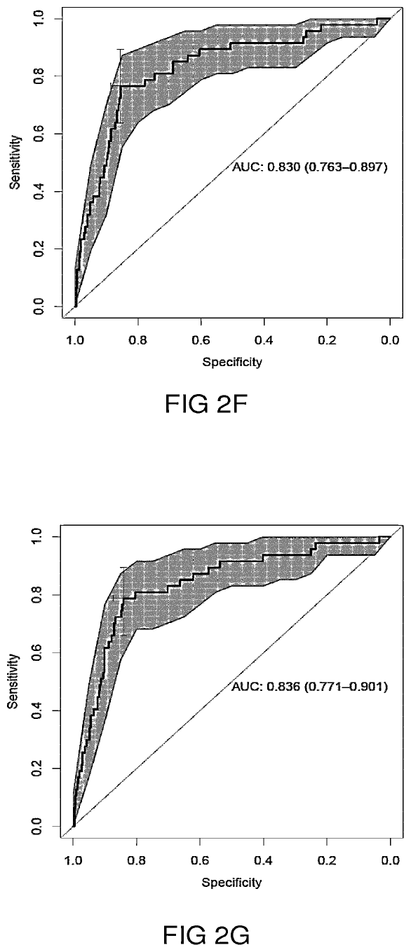

Generally, the quality of the prediction model can be increased by increasing the total number of marker substances included in the prediction model. FIG. 2A shows an ROC plot of the model for determining the risk of kidney rejection taking into account lactate, citrate, trigonelline and glucose (comparative example). Using these four already known substances as marker substances, an AUC of 0.732 results.

By using hippurate instead of trigonelline, the AUC drops to 0.717, as shown in FIG. 2B (comparative example).

When using a combination of lactate, citrate, trigonelline and hippurate as marker substances in a model for determining the risk of kidney rejection, an AUC of 0.716 results, as shown in FIG. 2C (comparative example).

Thus, a model making use of four already known marker substances results in an AUC ranging from 0.716 to 0.732.

The performance of an according model for predicting the risk of kidney rejection can be significantly increased by including two newly identified marker substances into the model. If the model is based on lactate, citrate, urea and alanine, an AUC of 0.752 results, as shown in FIG. 2D.

A similar high AUC can be observed for a model taking into account lactate, citrate, urea and glucuronate. This is shown in FIG. 2E, indicating an AUC of 0.748.

The prediction quality of an according model for determining the risk of kidney rejection after kidney transplantation can be even more increased if additional patient information is included in the model. FIG. 2F shows an ROC plot of a model taking into account lactate, citrate, urea and alanine as marker substances (like the model underlying FIG. 2D). By additionally considering the creatinine concentration in the blood serum of the same individual, the AUC can be increased from 0.752 (cf. FIG. 2D) to 0.830, as shown in FIG. 2F. Thereby, the calculated score for the predicted risk of kidney rejection has been multiplied by the concentration of creatinine in the blood serum of the same individual, a urine sample of whom has been analyzed.

A likewise high performance increase can be observed when combining a prediction model with additional information based on the estimated glomerular filtration rate (eGFR). FIG. 2G shows the performance of a model taking into account lactate, citrate, urea and alanine as marker substances (cf. FIG. 2D) and additionally considering the estimated glomerular filtration rate as further parameter. In doing so, the AUC increases from 0.752 (cf. FIG. 2D) to 0.836, as indicated in FIG. 2G. Thereby, the score for predicting the risk of kidney rejection calculated on the basis of lactate, citrate, urea and alanine has been divided by the determined estimated glomerular filtration rate of the same individual, a urine sample of whom has been analyzed.

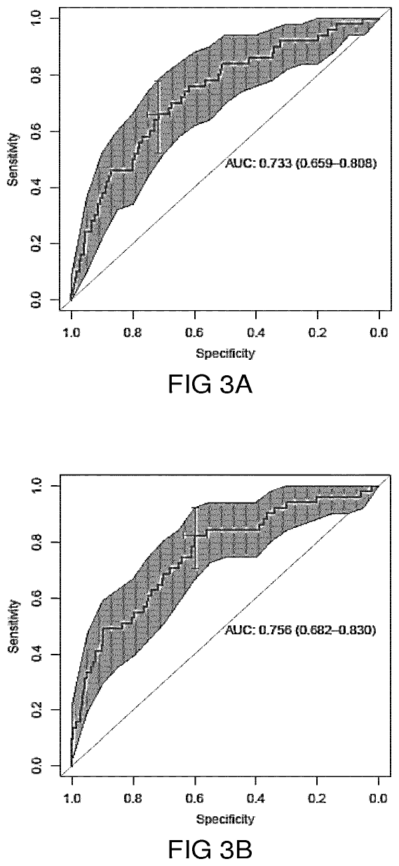

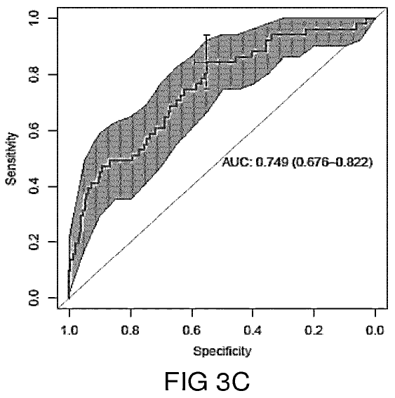

If five instead of four already known marker substances are used in a marker set of a model for predicting the risk of kidney rejection after kidney transplantation, the AUC can only hardly be increased. This is shown in FIG. 3A depicting an ROC plot of a model taking into account lactate, citrate, trigonelline, hippurate and glucose as marker substances (comparative example). The resulting AUC is 0.733 and thus only slightly higher than the AUC of a model taking into account lactate, citrate, trigonelline and glucose as marker substances (cf. FIG. 2A).

If, however, two already known marker substances (lactate and citrate) are combined with three newly identified substances, the prediction quality of the model can be significantly increased, as indicated by a significantly increased AUC. Specifically, if a model is used for predicting the risk of kidney rejection after kidney transplantation that takes into account lactate, citrate, urea, alanine and glucuronate as marker substances, an AUC of 0.756 results, as indicated in FIG. 3B.

If glucuronate is replaced by dimethylamine, the resulting AUC is 0.749 and thus slightly lower, but still much higher than without these newly identified marker substances. An according ROC plot is shown in FIG. 3C.

Summarizing, the newly identified marker substances alanine, dimethylamine, glucuronate, phenylacetylglutamine, and urea strongly increase the reliability and performance of prediction models for assessing the risk of kidney rejection after kidney transplantation if used alone or in combination with already known marker substances for this purpose. Thus, by the (additional) use of these marker substances, methods for determining or predicting the risk of kidney rejection after kidney transplantation can be performed with a much higher accuracy and reliability. This is likewise true for methods providing original data that can subsequently be used by a physician to make a diagnosis regarding the kidney rejection risk.

1. An in vitro method for determining the risk of kidney rejection of an individual after having obtained a kidney donation, the method comprising:
determining, with an NMR spectrometer, the concentration of at least three substances of a marker set in a urine sample of a patient, the concentration of substances derived from NMR spectra obtained from the NMR spectrometer;
analyzing the NMR spectra by subjecting the NMR spectra to automatic referencing, phase correction, and baseline correction;
calculating the risk of kidney rejection of the individual using the determined concentration; and
outputting the calculated risk of kidney rejection to the individual or to a third person, the marker set comprising at least three substances chosen from the group consisting of 3-hydroxy isovalerate, acetyl carnitine, alanine, citrate, dimethylamine, glucose, glucuronate, hippurate, lactate, malonate, methyl guanidine, methyl malonate, methyl succinate, p-cresol, phenyl acetate, phenylacetylglycine, phenylacetylglutamine, taurine, trigonelline, and urea, wherein the at least three substances of the marker set includes at least one substance chosen from the group consisting of alanine, dimethylamine, glucuronate, and phenylacetylglutamine,
wherein calculating the risk comprises multiplying each of the concentrations of the substances by a substance-specific weighting factor to provide a plurality of weighted values and combining the weighted values into a risk equation, followed by comparing an output of the risk equation to a predefined threshold, and
wherein outputting the calculated risk comprises presenting the calculated risk on a display.

2. The method according to claim 1, wherein the marker set comprises at least one substance chosen from the group consisting of alanine, dimethylamine, and glucuronate.

3. The method according to claim 1, wherein the marker set comprises urea.

4. The method according to claim 1, wherein the marker set comprises lactate and citrate.

5. The method according to claim 1, wherein the marker set does not comprise dimethylamine, lactate, acetate and alanine at the same time.

6. The method according to claim 1, wherein the marker set consists of dimethylamine, lactate, alanine, and at least one substance of the group consisting of 3-hydroxy isovalerate, acetyl carnitine, citrate, glucose, glucuronate, hippurate, malonate, methyl guanidine, methyl malonate, methyl succinate, p-cresol, phenyl acetate, phenylacetylglycine, phenylacetylglutamine, taurine, trigonelline, and urea.

7. A method for analyzing an isolated body fluid sample in vitro, comprising the following steps:
a) providing a urine sample from an individual who has received a kidney donation,
b) determining, with an NMR spectrometer, the concentration of at least three substances chosen from the group consisting of 3-hydroxy isovalerate, acetyl carnitine, alanine, citrate, dimethylamine, glucose, glucuronate, hippurate, lactate, malonate, methyl guanidine, methyl malonate, methyl succinate, p-cresol, phenyl acetate, phenylacetylglycine, phenylacetylglutamine, taurine, trigonelline, and urea in the body fluid sample by analyzing the body fluid sample with a spectrometer, where the concentration of at least one substance chosen from the group consisting of alanine, dimethylamine, glucuronate, and phenylacetylglutamine is determined, the concentration of substances derived from NMR spectra obtained from the NMR spectrometer;
c) analyzing the NMR spectra by subjecting the NMR spectra to automatic referencing, phase correction, and baseline correction;
d) calculating a score from the determined concentrations by multiplying each of the concentrations of the substances by a substance-specific weighting factor to provide a plurality of weighted values and combining the weighted values into a risk equation, the score being indicative for the risk of kidney rejection of the individual,
e) determining a specific risk of kidney rejection using the calculated score and a predefined allocation of individual scores to specific risks of kidney rejection, and
f) outputting, with a display, at least one of the score and the specific risk of kidney rejection to the individual or a third person.

8. The method according to claim 7, wherein the determined concentrations are normalized to the concentration of creatinine in the same sample.

9. The method according to claim 7, wherein the sample is grouped into one of at least two predefined groups on the basis of the calculated score.

10. The method according to claim 7, wherein an extended score is calculated from the score by additionally considering at least one value of the group consisting of a value being indicative for the concentration of creatinine in blood serum of the individual, a value being indicative for the glomerular filtration rate of the individual, and a value being indicative for the estimated glomerular filtration rate of the individual.

11. The method according to claim 10, wherein the sample is grouped into one of at least two predefined groups on the basis of the calculated extended score.

12. The method according to claim 1, wherein the step of calculating risk comprises an area-under-the-curve calculation of the weighted values.

13. The method according to claim 7, wherein the step of determining a specific risk of kidney rejection comprises an area-under-the-curve calculation of the weighted values.

14. The method according to claim 7, wherein the step of analyzing the NMR spectra further comprises automatically standardizing and calibrating the NMR spectra.

15. The method according to claim 7, wherein the step of analyzing the NMR spectra occurs automatically following the step of determining the concentration of the at least three substances.
